(19) United States
(12) Patent Application Publication
(10) Pub. No.: US 2004/0265431 A1
(43) Pub. Date: Dec. 30, 2004

(54) Use of a single-cell protein as feed for fish and shellfish

(76) Inventor: Gunnar Kleppe, Hafrsfjord (NO)

Correspondence Address: SUGHRUE MION, PLLC, 2100 PENNSYLVANIA AVENUE, N.W., SUITE 800, WASHINGTON, DC, 20037, US

(21) Appl. No.: 10/486,693

(22) PCT Filed: Aug. 16, 2002
(86) PCT No.: PCT/GB02/03795

(30) Foreign Application Priority Data
Aug. 16, 2001 (GB) 0120047.6

(51) Int. Cl. A23L 1/28; A23C 9/12; C12N 1/20

(52) U.S. Cl. 426/61; 435/252.1

(57) ABSTRACT

The invention relates to a feed material for fish and shellfish, for example a feed material for molluscs such as mussels. In particular, the invention relates to the use of single-cell protein materials and their derivatives (especially homogenized and/or autolysed derivatives) as a feed for fish and shellfish. A preferred single-cell material for use in the invention is that derived from a microbial culture comprising Methylococcus capsulatus (Bath) (strain NCIMB 11132), Ralstonia sp. DB3 (strain NCIMB 13287) and Brevibacillus agri DB5 (strain NCIMB 13289), optionally in combination with Aneurinibacillus sp. DB4 (strain NCIMB 13288).

The present invention relates to a feed material for fish and shellfish, for example a feed material for molluscs such as mussels. In particular, the invention relates to the use of single-cell protein materials and their derivatives as a feed for fish and shellfish.

Single-cell microorganisms such as fungi, yeasts and bacteria have been proposed as new sources of protein in human foods and as certain animal feeds, e.g. feeds for chickens, pigs, etc. These may be grown by single-cell reproduction and several bio-synthetic processes for the production of protein through the growth of single-cell microorganisms on hydrocarbon and other substrates have been developed. For example, PCT/GB01/00628 (published as WO 01/60974), DK 1404/92, EP-A-418187 and EP-A-306466 describe various methods and apparatus suitable for the production of such materials. The entire contents of each of these documents is incorporated herein by reference.

It has now been found that protein-containing microorganisms (also referred to herein as “single-cell proteins”) and derivatives thereof, especially homogenized and autolysed derivatives, may be used as fish and shellfish feed. Particularly effective fish and shellfish feeds may be produced using the biomass harvested from a culture medium comprising methanotrophic bacteria, e.g. biomass produced as described in WO 01/60974, and derivatives thereof.

Viewed from one aspect the invention thus provides the use of a single-cell protein material or derivative thereof (e.g. a homogenized or autolysed derivative of a single-cell protein material) as a fish or shellfish feed or feed additive.

In a further aspect the invention provides the use of a single-cell protein material or derivative thereof (e.g. a homogenized or autolysed derivative of a single-cell protein material) in the manufacture of a fish or shellfish feed, or in the manufacture of a fish or shellfish feed additive.

As used herein the term “derivative” when used in relation to a single-cell protein material, e.g. a microbial culture, includes any product which may be derived from such a material using a downstream processing technique or techniques (e.g. a series of techniques) known in the art, such as separation of a single-cell protein material from a fermentation medium or liquid by centrifugation and/or ultrafiltration methods, or by dewatering, etc. such techniques also include those which may be used to break down the is single-cell material (e.g. the bacterial cells), such as homogenization and/or autolysis.

Ad used herein, the term “homogenized” when used in relation to a single-cell protein material is intended to define such a material in which a substantial proportion (e.g. substantially all) of the cells are broken down, i.e. disrupted or disintegrated, e.g. as a result of mechanical disruption. Such homogenized materials will generally consist of a viscous protein slurry containing both soluble and particulate cellular components.

The term “autolysis” as used herein is intended to encompass a process in which endogenous enzymes contained within a cell, such as nucleases and proteases, digest the components of the cell.

A preferred aspect of the invention relates to the use of the products herein described as a feed material for shellfish, for example in shellfish cultivation. Examples of shellfish to which the products herein described may be fed include mussels, oysters, scallops, etc.

Surprisingly, it has been found that the products herein described are particularly effective in improving (e.g. increasing the rate of) detoxification or decontamination of shellfish (especially mussels) contaminated by toxic algae. For example, these may be used to detoxify toxic algae-containing shellfish to levels that are non-toxic to human beings.

In a yet further aspect the invention thus provides a method of detoxifying shellfish (e.g. mussels), said method comprising supplying to said fish an effective amount of a single-cell protein material or derivative thereof as herein described.

The product herein described may also be used in feeding marine fish, particularly white fish such as cod. For example, this may find use as a feed for fish which are bred for human consumption and may therefore be used in fish farming.

Other fish which may be fed with the material herein described are those kept in aquariums. Such fish include those kept for exhibition or as pets for pleasure, e.g. goldfish, koi carp, tropical fish, etc.

Preferred single-cell protein materials for use in the invention are homogenized single-cell protein materials such as those described in PCT/GB01/00628 (WO 01/60974), the entire contents of which are incorporated herein by reference. However, the basic single-cell protein material described in this reference (i.e. that which has not been subjected to homogenization) may also be used. In this regard, although the material produced in the fermentor may be directly used as the feed material (or feed additive) without further processing, this will generally be subjected to additional downstream processing steps, such as for example centrifugation and/or filtration (e.g. ultrafiltration) processes. Both homogenized and non-homogenized materials will typically be subjected to dewatering (e.g. spray drying) and optionally sterilization prior to use as a feed material or feed additive. A dried homogenizate of the bacterial biomass described in WO 01/60974 is a particularly preferred material for use in accordance with the present invention.

Single-cell protein materials, e.g. bacterial cultures, which have been subjected to autolysis to break down the cells are also suitable for use in the invention. Such a treatment and the resulting autolysates are described, for example, in British Patent Application No. 0203307.4 filed 12 Feb. 2002, the content of which is incorporated herein by reference (copy attached hereto). The autolysate, more especially the dried autolysate, described in this earlier application is considered particularly suitable for the preparation of feed materials in accordance with the invention.

Particularly preferably, the single-cell protein material for use in the invention will be derived from a microbial culture which comprises at least one methanotrophic bacteria optionally in combination with one or more species of heterotrophic bacteria, especially preferably a combination of methanotrophic and heterotrophic bacteria. Preferably these will be grown in the same culture medium, e.g. using a loop reactor provided with methane, oxygen, ammonia and mineral feeds. Such materials and processes for their preparation are described in PCT/GB01/00628 (WO 01/60974) the entire contents of which are incorporated herein by reference. One particularly suitable combination of bacteria for generating a biomass for use in the invention is Methylococcus capsulatus (Bath) (strain NCIMB 11132), in combination with Ralstonia sp. DB3 (strain NCIMB 13287) and Brevibacillus agri DB5 (strain NCIMB 13289), optionally together with Aneurinibacillus sp. DB4 (strain NCIMB 13288).

The bacterial culture used to produce the biomass is preferably at least 50%, more preferably at least 60%, especially at least 70%, in particular at least 75%, e.g. 75 to 95%, more particularly 75 to 80%, by weight methanotrophic bacteria (relative to the total bacterial weight).

As used herein, the term “methanotrophic” encompasses any bacterium which utilizes methane, methanol or formaldehyde for growth. The term “heterotrophic” is used for bacteria that utilize organic substrates other than methane, methanol or formaldehyde for growth.

The bacterial biomass for use in the invention may be formed by growth of the bacteria on a suitable medium or substrate. The exact nature of the growth medium used to produce the biomass is not critical and a variety of suitable substrates may be used. Conveniently, the biomass may be produced by a fermentation process in which oxygen and a suitable substrate such as a liquid or gaseous hydrocarbon, an alcohol or carbohydrate, e.g. methane, methanol or natural gas, together with a nutrient mineral solution are fed to a tubular reactor containing the microorganisms. Such a process is described, for example, in WO 01/60974.

A particularly preferred biomass material for use in the present invention is that derived from fermentation on hydrocarbon fractions, particularly preferably that derived from fermentation on natural gas.

For use as a fish or shellfish feed, appropriate amounts of the material herein described will depend on several factors, such as the species of fish or shellfish for which the product is intended. However, such amounts can readily be determined by those skilled in the art.

For most applications, especially for feeding shellfish, the feed will be supplied in particulate form (e.g. as granules or in powder form). For feeding shellfish, the particles will typically be added to seawater which is then circulated through the holding tanks. Suitable particle sizes may range from 0.5 to 20 μm. However, feeds in the form of a liquid (e.g. as an aqueous slurry) may also be used and are considered suitable for use in feeding shellfish, especially shellfish which are attached to a culture rope.

In a still further aspect the invention thus provides a liquid feed for shellfish (e.g. mussels), said feed comprising an aqueous slurry of a single-cell protein or derivative thereof as herein described.

The invention will now be described in more detail by way of the following non-limiting Examples:

### EXAMPLE 1

### Biomass Extracts

Methanotrophic and heterotrophic bacteria (Methylococcus capsulatus (Bath) (strain NCIMB 11132), Ralstonia sp. DB3 (strain NCIMB 13287), Brevribacillus agri DB5 (strain NCIMB 13289), and Aneurinibacillus sp. DB4 (strain NCIMB 13288)) are cultivated as described in WO 01/60974 and the resulting biomass harvested and treated as described in WO 01/60974 to produce a spray-dried, particulate homogenizate, and as described in British Patent Application No. 0203307.4 to produce a spray dried autolysate.

### EXAMPLE 2

### Feeding Shellfish

The homogenizate produced in Example 1 is suspended in sea water at a concentration of from 10 to 1,000 ppm. This suspension of feed is then circulated through a tank containing shellfish (e.g. mussels, oysters or scallops) for a period of from 1 to 3 weeks.

The same feeding regimen may be carried out utilising the autolysate material of Example 1.

1-8. (Cancelled).

9. A method for preparing a fish or shellfish feed or feed additive comprising a homogenizing or autolysing a single-cell protein material so as to obtain said feed or feed additive, and optionally, adding the resulting feed additive to a fish or shellfish feed.

10. The method as claimed in claim 9, wherein said single-cell protein material is derived from the biomass of a culture of bacteria comprising methanotrophic bacteria, and optionally at least one species of heterotrophic bacteria.

11. The method as claimed in claim 10, wherein said culture comprises Methylococcus capsulatus (Bath) (strain NCIMB 11132), Ralstonia sp. DB3 (strain NCIMB 13287) and Brevibacillus agri DB5 (strain NCIMB 13289), optionally in combination with Aneurinibacillus sp. DB4 (strain NCIMB 13288).

12. The method as claimed in claim 10, wherein said culture is produced by fermentation on hydrocarbon fractions or on natural gas.

13. The method as claimed in claim 10, wherein said culture is produced from fermentation on natural gas.

14. A method of detoxifying shellfish comprising supplying to said shellfish an effective amount of a single-cell protein material or derivative thereof.

15. The method as claimed in claim 14, wherein said derivative is a homogenized or autolysed derivative of a single-cell protein material.

16. The method as claimed in claim 14, wherein said shellfish are mussels.

17. The method as claimed in claim 14, wherein said single-cell protein material is derived from the biomass of a culture of bacteria comprising methanotrophic bacteria, and optionally at least one species of heterotrophic bacteria.

18. The method as claimed in claim 17, wherein said culture comprises Methylococcus capsulatus (Bath) (strain NCIMB 11132), Ralstonia sp. DB3 (strain NCIMB 13287) and Brevibacillus agri DB5 (strain NCIMB 13289), optionally in combination with Aneurinibacillus sp. DB4 (strain NCIMB 13288).

19. The method as claimed in claim 17, wherein said culture is produced by fermentation on hydrocarbon fractions or on natural gas.

20. The method as claimed in claim 17, wherein said culture is produced from fermentation on natural gas.

21. A method for preparing a shellfish feed or shellfish feed additive comprising obtaining a biomass of a culture of bacteria comprising Methylococcus capsulatus (Bath) (strain NCIMB 11132), Ralstonia sp. DB3 (strain NCIMB 13287) and Brevibacillus agri DB5 (strain NCIMB 13289), optionally in combination with Aneurinibacillus sp. DB4 (strain NCIMB 13288), and preparing a single-cell protein material therefrom, so as to obtain said feed or feed additive, and optionally, adding the resulting feed additive to a shellfish feed.

22. A method of feeding fish or shellfish, said method comprising the step of supplying to said fish or shellfish a homogenized or autolysed derivative of a single-cell protein material.

23. A method of feeding shellfish, said method comprising the step of supplying to said shellfish a single-cell protein material which is derived from the biomass of a culture of bacteria comprising Methylococcus capsulatus (Bath) (strain NCIMB 11132), Ralstonia sp. DB3 (strain NCIMB 13287) and Brevibacillus agri DB5 (strain NCIMB 13289), optionally in combination with Aneurinibacillus sp. DB4 (strain NCIMB 13288).
